(19) United States
(12) Patent Application Publication
(10) Pub. No.: US 2003/0119190 A1
(43) Pub. Date: Jun. 26, 2003

(54) Genetic tagging strategy for inducing and identifying mutations in a genomic sequence

(76) Inventors: Clifford Lee Wang, Berkeley, CA (US); Matthias Wabl, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/265,175

(22) Filed: Oct. 3, 2002

Related U.S. Application Data
(60) Provisional application No. 60/327,129, filed on Oct. 3, 2001.

(51) Int. Cl. C12N 15/85; C12Q 1/68; C12P 19/34; C12N 5/06

(52) U.S. Cl. 435/455; 435/91.2; 435/6; 435/326

(57) ABSTRACT

The invention features methods and compositions for introducing mutations in an endogenous host cell gene. In one embodiment, the invention features identification of genes that, when mutated, result in production of a phenotype of interest, e.g., tumor formation. In general, the invention provides a random mutagenesis system wherein a non-oncogenic, replicating vector acts as a vehicle to randomly introduce a construct comprising a hypermutation-inducing element into the genome of a host cell. Introduction of the hypermutation element in the host cell genome induces mutations (e.g., point mutations, small deletions, and/or small insertions) in genes adjacent to the integrated hypermutation element.

### CROSS-REFERENCE TO RELATED APPLICATIONS

This application claims the benefit of prior U.S. Provisional Application Serial No. 60/327,129, filed Oct. 3, 2001, which application is incorporated herein by reference.

### GOVERNMENT RIGHTS

The United States Government may have certain rights in this application pursuant to NIH Grants GM 37699 and AG 20684.

### FIELD OF THE INVENTION

The invention relates to methods for generating mutations in a gene and identifying such mutations that are associated with phenotypic changes, such as tumor formation.

### BACKGROUND OF THE INVENTION

Many methods have been developed to facilitate the study of mutations at the genetic level and to expedite the identification of genetic mutations, which in many cases, will lead to oncogenesis. A particular challenge is the identification of small changes in genes, such as point mutations, that are associated with oncogenesis. One method of identifying oncogenic mutations involves a series of linkage analyses of markers among a selected population of cancer patients, genetic mapping to a chromosome, positional cloning, and functional analysis and sequencing of resulting candidate genes. An alternative method involves the genomic analysis of families predisposed to a certain type of cancer to identify sequences consistently deleted, amplified, or translocated using interphase and/or metaphase fluorescent in situ hybridization (FISH), conventional cytogenetics, or comparative genomic in situ hybridization (CGH). However, these methods are cumbersome; furthermore, FISH and CGH are generally limited to detection of large chromosomal abnormalities.

Transgenic animal models have been established in the art as a tool for studying gene function in vivo, and have served to aid in the study of the effects of gene copy number, overexpression, and/or sequence changes upon tumorigenesis. Generation of mutated genes for use in generation of transgenic animals can be accomplished using any of a variety of in vitro techniques, such as site-directed mutagenesis, saturation mutagenesis, or scanner mutagenesis.

In site-directed mutagenesis, the gene encoding the protein of interest is cloned into a prokaryotic vector, and mutations are introduced by hybridizing the gene with an oligonucleotide that contains the desired mutation, followed by a replication event that completes the synthesis of the new, mutated gene. In saturation mutagenesis the gene is altered randomly. In so-called scanner mutagenesis, a subfraction of codons encoding a particular amino acid residue is replaced, e.g., by alanine. In still another approach the error rate of polymerase chain reaction (PCR) is used to introduce mutations; the error rate is due to the intrinsic infidelity of the polymerase used in PCR and can be increased by altering the ratio of nucleotide concentrations. Mutagenesis in Escherichia coli can be carried out using mutator strains, e.g., strains defective in DNA repair. However, these approaches are impractical on a genome-wide scale. Furthermore, generation of transgenic models requires that one have in hand a candidate gene, and thus such techniques cannot be used for gene discovery.

Another approach involves generating mutations in vivo, e.g., by exposing an animal to a carcinogenic insult (e.g., chemicals, radiation, environmental insults such as exhaust, and the like). See, e.g., Myers et al. (1986) Science 232:613-618. A chemical method of random mutagenesis, for example, consists of including exposure of the host to DNA intercalating compounds such as nitrosoguanidine and acridine orange, or commonly used mutagens such as ENU and sodium bisulfate. However, exposure to such carcinogens and mutagens often results in mutation of multiple genes. Furthermore, identification of the genes mutated by exposure to these substances is not a simple matter, and generally requires breeding. While this approach is advantageous in that it is essentially random, at the present time it leaves no direct means for readily identifying the gene or genes mutated.

Retroviruses such as MMTV, MLV, and HTLV are commonly used in the art for generating tumors by promoting abnormal cell proliferation. Tumor formation can result due to retroviral insertional mutagenesis, wherein transcription of neighboring genes in the native genome is affected (see, e.g., Sourvinos et al. (2000) Folia Biol. (Praha) 46(6):226-232). This mode of tumor induction is frequently adopted using retroviruses lacking oncogenes. In some cases, like for the Fgf-3 protooncogene, both a change in coding sequence and upregulation of transcription occurs as a result of insertional mutagenesis. See, Morris et al. (1997) Virology 238(1):161-165; Varmus (1983) Prog. Clin. Biol. Res. 119:23-25; and Nusse et al. (1982) Cell 31(1):99-109. The integrated retroviral sequences facilitate identification of mutations that lead to tumor development, or other phenotypic changes. However, the use of these viruses is limited in that viral integration usually involves increased transcription or disruption of genes, and is not very useful in the identification of small changes such as point mutations that can lead to tumorigenesis, and other diseases characterized by DNA mutations.

There are many types of mutations that change not the just the structure of the protein itself, but can change where and how much of a protein is made. These types of mutations in the DNA can result in aberrant protein expression. The protein is made at the wrong time or in the wrong cell type. Changes can also occur that result in too much or too little of the protein being made. These changes do not always result in diminished cellular function. Mutagenesis can improve the affinity of a receptor, for example, or alter the efficiency of a cellular process.

The hypervariable regions of the immunoglobulin (Ig) locus is an example of a naturally occurring site directed mutagenesis at a high rate. The hypervariable regions are the regions encoding the heavy or light chain polypeptides, in which there is considerable sequence diversity within that set of immunoglobulins in a single individual. These regions specify the antigen affinity of each antibody and in turn provide the immune system with the ability to generate antibodies that specifically bind a wide variety of epitopes. Somatic point mutations in the variable regions of the antibody light and heavy chains are a major source of this antibody diversity. See, e.g., Gearhart et al. (1981) Nature 291:29-34; Bothwell et al. (1981) Cell 24:625-637; Crews et al. (1981) Cell 25:59-70; Kim et al. (1981) Cell 27:573-581; Selsing et al. (1981) Cell 25:47-58; Gearhart et al. (1983) Proc. Natl. Acad. Sci. U.S.A. 80:3439-3443; McKean et al. (1984) Proc. Natl. Acad. Sci. U.S.A. 81:3180-3184; Manser et al. (1984) Science 226:1283-1288; Rudikoff et al. (1984) Proc. Natl. Acad. Sci. U.S.A. 81:2162-2165; Berek et al. (1985) Nature 316:412-418; Clarke et al. (1985) J. Exp. Med. 161:687-704; Sablitzky et al. (1985) EMBO J. 4:345-350; and Griffiths et al. (1984) Nature 312:271-275.

Because of the high frequency of mutations, the process that produces somatic mutations in the hypervariable regions is referred to as “hypermutation.” Hypermutation is active during B-cell proliferation after antigenic stimulation. Indeed, cells undergoing hypermutation are found in the germinal centers, where the B-cell response in large part takes place (Jacob et al. (1991) Nature 354:389-3892; and Ziegner et al. (1994) Eur. J. Immunol. 24:2393-2400).

At the heavy chain locus, a stretch of about 2 kb, which includes the rearranged V(D)J segments and their flanking regions, is especially hypermutable. The 5′ hypermutation boundary near the promoter region is sharp; the 3′ boundary near the enhancer region is less well defined. Within the hypermutable sequences, some sites are referred to as “hot spots,” for which consensus sequence motifs are known, and which are intrinsically more mutable than neighboring sequences (Rogozin et al. (1992) Biochim. Biophys. Acta 1171:11-18). The V(D)J sequence itself is not needed to trigger the process (Yelamos et al. (1995) Nature 376:225-229). When the V(D)J sequence is replaced by another non-Ig sequence that sequence becomes hypermutable (Yelamos et al. (1995) supra).

The segments encoding the immunoglobulin variable (V) region is the ‘epicenter’ of mutation, with the frequency of mutation decreasing progressively in both 5′ and 3′ directions. The area of optimal hypermutation of about 2 kb includes the flanking regions.

Hypermutation requires a promoter, but not necessarily the Ig-gene promoter. Rather, it is only important that transcription occurs (Tumas-Brundage et al. (1997) J. Exp. Med. 185:239-250; Fukita et al. (1998) Immunity 9:105-114; and Burke et al. (2001) J. Immunol. 166:5051-5057). An enhancer 3′ of, and at some distance from the hypermutation site is also needed, and this enhancer need not be a sequence from an Ig locus. For the kappa (light chain) and heavy chain loci, it has been shown that the intronic enhancer is necessary (Betz et al. (1994) Cell 77:239-248; Bachl et al. (1998) Proc. Natl. Acad. Sci. U.S.A. 95:2396-2399; Bachl and Olson. (1999) Eur. J. Immunol. 29: 1383-1389; Bachl et al. (2001) J. Immunol. 166: 5051-5057). The intronic enhancer is the only sequence that is required from the Ig locus. Although transcriptional enhancement by the intronic enhancer is orientation independent, hypermutation is orientation dependent for the most effective hypermutation (Bachl et al. (1998) Proc. Natl. Acad. Sci. U.S.A. 95:1296-2399). Both promoter and enhancer are most effective when they are provided at an appropriate distance from the sequence to be mutated. See, Tumas-Brundage et al. (1997) supra; Bachl et al. (1998), supra; and Winter et al. (1997) Mol. Immunol. 34:359-366. For additional information on the cis-acting elements of the hypervariable region and other factors affecting hypermutation, see, e.g., Wabl et al. (1999) Curr. Op. Immunol. 11:186-189; Lebecque et al. (1990) J. Exp. Med. 172:1717-1727; Weber et al. (1991) J. Immunol. 146:3652-3655; Rogerson et al. (1994) Mol. Immunol. 31:83-98; Rada et al. (1994) Eur. J. Immunol. 24:1453-1457; Gonzalez-Fernandez et al. (1994) Proc. Natl. Acad. Sci. 91:12614-12618; Betz et al. (1994) Cell 77:239-248; Sharpe et al. (1991) EMBO J. 10:2139-2145; Rogerson et al. (1991) EMBO J. 10:4331-4341; Sharpe et al., in: Fundamental Immunology, Chapter 10, Third Edition, William Paul, 1993; Betz et al. (1993) Proc. Natl. Acad. Sci. U.S.A. 90:2385-2388; Yelamos et al. (1995) Nature 376:225-229; and U.S. Pat. No. 5,371,009.

Neuberger et al. in U.S. Pat. No. 5,371,009, describe a functional dissection of the kappa 3′ enhancer, which is a “non-specific” transcriptional enhancer, and identified a 53 bp region responsible for enhanced transcription. Neuberger et al. also describe hypermutation experiments conducted in a transgenic mouse comparing constructs that did or did not include the 8 kb region 3′ of Cₖ, which contains the entire 3′ kappa enhancer, but do not describe use of the 53 bp fragment alone. In addition, the transgene used by Neuberger et al. encoded an antibody directed against phOX; no hypermutation of an endogenous host gene or of a non-immunoglobulin gene is described.

Yelamos et al. (1995) Nature 376:225-229, describe production of a transgene by replacement of the V gene segment in a light chain with bacterial sequences encoding gpt and neomycin resistance (Neo®). The bacterial sequences in the construct, which contained other elements needed for hypermutation (a promoter, the light chain major intron enhancer and 3′ enhancer), underwent hypermutation during an immune response. Azuma et al. (1993) Intl. Immunol. 5(2):121-130, inserted the heterologous CAT reporter gene into a heavy chain gene, and the resulting construct was used to create a transgenic mouse for subsequent evaluation of hypermutation in vivo. Likewise, Umar et al. (1991) Proc. Natl. Acad. Sci. U.S.A. 88:4902-4906, inserted a heterologous reporter gene into a rearranged kappa gene and used the product to create a transgenic mouse for subsequent evaluation of hypermutation in vivo. Johnston et al. (1992) Mol. Immunol. 29(708):1005-1011, developed a shuttle vector system for the investigation of Ig gene hypermutation and observed an absence of enhanced mutability in intermediate B cell lines transfected with a VD segment and a portion of the heavy chain intron.

The hypermutation system of the Ig locus has been exploited to provide a method for in vitro saturation mutagenesis. U.S. Pat. No. 5,885,827 describes an in vitro system that can be used to generate point mutations in a cloned gene. However the system still requires that one have a candidate gene cloned. Thus, this system, while helpful in evaluating known candidate genes, is not useful in the identification of new genes and new mutations that can cause oncogenesis and other phenotypic changes arising from point mutations.

To date there has been no system that provides for essentially random, but tagged mutagenesis of genes in vivo, in the cell or in the animal, where mutagenesis provides for introduction of point mutations. There is a need for methods and compositions that provide for both these phenomena while providing a means for readily identifying the mutated gene. The present invention addresses these needs.

### SUMMARY OF THE INVENTION

The invention features methods and compositions for introducing mutations in an endogenous host cell gene. In one embodiment, the invention features identification of genes that, when mutated, result in production of a phenotype of interest, e.g., tumor formation. In general, the invention provides a random mutagenesis system wherein a non-oncogenic, replicating vector acts as a vehicle to randomly introduce a construct comprising a hypermutation-inducing element into the genome of a host cell. Introduction of the hypermutation element in the host cell genome induces mutations (e.g., point mutations, small deletions, and/or small insertions) in genes adjacent to the integrated hypermutation element.

In one aspect the invention features a method for mutating a gene in a host cell genome, the method comprising introducing a hypermutation-inducing construct into a vertebrate host cell, the construct comprising a cis-acting hypermutation element, wherein introducing of the construct provides for integration of at least the cis-acting hypermutation element into a host cell genome and adjacent an endogenous host cell gene so that transcription of the endogenous host gene and the cis-acting hypermutation element facilitates introduction of a mutation into the endogenous host gene to generate a mutated gene.

In specific embodiments, the hypermutation inducing construct comprises at least one immunoglobulin intronic enhancer. In further specific embodiments, the immunoglobulin intronic enhancer is a heavy chain large intronic enhancer or a kappa intronic enhancer. In still further specific embodiments, the method further comprises identifying the mutated gene adjacent the cis-acting element, with detecting being, for example, by detecting all or a portion of integrated construct or by amplification of at least a portion of a genomically integrated portion of the construct and a portion of the adjacent mutated gene to produce an amplification product comprising a sequence of the adjacent mutated gene. In still further embodiments, the mutated gene is associated with a cellular phenotype of interest, exemplified by an oncogenic phenotype. In further embodiments, the host cell is a cultured cell or cell line, or is present in a non-human animal (e.g., a murine non-human animal).

In another aspect, the invention features a method for identifying a proto-oncogene, which gene becomes oncogenic upon introduction of a point mutation, the method comprising introduction of a hypermutation inducing construct into a non-human animal host, or into any animal cell, including human, the construct comprising a cis-acting hypermutation element, wherein said cis-acting hypermutation element comprises an immunoglobulin intronic enhancer. Introduction of the construct provides for integration of at least the cis-acting hypermutation element into a host cell genome and adjacent an endogenous host gene so that transcription of the endogenous host gene and the cis-acting element facilitates production of a mutated host gene having point mutations. Tumor formation is then detected in the host, and the mutated gene adjacent the cis-acting hypermutation element in nucleic acid of the tumor is identified. Detection of a tumor having a mutated endogenous gene adjacent the hypermutation element indicates that the endogenous gene is a proto-oncogene.

In specific embodiments, the immunoglobulin intronic enhancer is a heavy chain large intronic enhancer or a kappa intronic enhancer. In further specific embodiments, the hypermutation inducing construct is contained in a viral vector, e.g,. a retroviral vector. In other embodiments, the mutated gene is identified by detecting all or a portion of integrated construct adjacent the mutated gene, or by amplification of at least a portion of a genomically integrated portion of the construct and a portion of the adjacent mutated gene to produce an amplification product comprising a sequence of the adjacent mutated gene. In further specific embodiments, the non-human host is a mouse.

In another aspect, the invention features a method for identification of a gene that becomes oncogenic after point mutation, the method comprising introducing a hypermutation inducing construct into a murine host, wherein the construct comprises a cis-acting hypermutation element, and wherein the cis-acting hypermutation element comprises a heavy chain large intronic enhancer or a kappa intronic enhancer. Introduction of the construct provides for integration of at least the cis-acting hypermutation element into a murine cell genome and adjacent an endogenous gene so that transcription of the endogenous gene and the cis-acting element facilitates production of a mutated gene having point mutations. Tumor formation is then detected, and the mutated gene adjacent the cis-acting hypermutation element in nucleic acid of the tumor identified. Detection of tumors formed as a result of mutation of the endogenous gene indicates that the endogenous gene is a proto-oncogene.

In specific embodiments, the hypermutation inducing construct is contained in a viral vector (e.g., a retroviral vector, such as a vector derived from Mouse Mammary Tumor Virus or Murine Leukemia Virus). In further specific embodiments, identification of the mutated gene is by detecting an integrated portion of the integrated construct, or by amplification of at least a portion of a genomically integrated portion of the construct and a portion of the adjacent mutated gene to produce an amplification product comprising a sequence of the adjacent mutated gene.

In yet another aspect, the invention features a vertebrate cell having a genomically integrated cis-acting hypermutation element, which element is adjacent and operably linked to a gene endogenous to the vertebrate cell and with which the element is not normally found in nature. In one embodiment, the hypermutation element is adjacent a gene other than an immunoglobulin gene or is adjacent a gene other than a reporter gene. In another specific embodiment, the cell is a cultured cell or cell line. In another embodiment, the cell is a mutator positive cell. In still another embodiment, the cell is an isolated vertebrate cell, or a vertebrate, non-human cell, containing a mutated gene produced by the method of the invention.

In another aspect, the invention features a non-human animal having a genomically integrated cis-acting hypermutation element, which element is adjacent and operably linked to a gene endogenous to the animal and with which the element is not normally found in nature. In a specific embodiment, the hypermutation element is adjacent a gene other than an immunoglobulin gene or a reporter gene.

In still another aspect, the invention features a vector comprising a cis-acting hypermutation element, wherein the cis-acting hypermutation element is not operably linked to a nucleic acid encoding a non-viral polypeptide (more specifically, the cis-acting hypermutation element is not operably linked to a gene encoding a reporter polypeptide or a immunoglobulin polypeptide), and wherein the vector is adapted for integration into a genome of a vertebrate cell. In specific embodiments, the vector is adapted for random integration in the vertebrate cell genome, and can be a viral vector, particularly a retroviral vector. In another embodiment, the cis-acting hypermutation element is an immunoglobulin intronic enhancer, such as a heavy chain intronic enhancer or a kappa chain intronic enhancer.

Thus, in general, the invention provides for introduction of a mutation, particularly a point mutation, a small deletion (e.g., 1, 2, or 3 nucleotides), and/or a small insertion (e.g., 1, 2, or 3 nucleotides) in a host cell genome. Of particular interest are mutations that are associated with an identifiable phenotypic change in the host cell or host animal. Nucleic acid from the host cell is then extracted for identification of the mutated gene.

In another aspect, the invention provides a eukaryotic mutagenesis system that operates in vivo (in an isolated cell or in a non-human animal, with production of mutations in genomic sequences in a living non-human animal being of particular interest) to induce mutations, particularly random mutations, more particularly random point mutations, small deletions, and/or small insertions.

The invention also provides, in another aspect, a system with a built-in tagging feature for ready identification of the mutated gene.

In still another aspect, the invention provides a system for identification of genes that, when mutated, are associated with tumor formation, thus facilitating the identification of genes with oncogenic potential (i.e., proto-oncogenes).

In yet another aspect, the invention provides a system for identification of genes that, when mutated, cause increased receptor affinity for a ligand or other biological phenomenon of interest, e.g., to identify modified genes with the potential to modify (increase or decrease) cell function.

One advantage of the invention is that mutations are introduced in genes in vivo (e.g., in their natural genomic setting) and with relatively little recombinant manipulation.

Another advantage of the invention is that the system provides a high mutation rate approaching or exceeding 10⁻⁴/base pair/cell generation.

Another advantage of the invention is that, unlike most conventional methods of inducing mutagenesis, no prior knowledge of the gene of interest, the host genetic sequence, or any clone of a gene of interest need to be known in advance.

A further advantage of the invention is that the system does not require an artificial selection process. For example, cells that have undergone hypermutation that renders a gene oncogenic will develop into tumors which can be isolated for analysis.

Still another advantage is that transfer of mutation-inducing factors into the host can be accomplished by simple procedures such as administration of a virus harboring the necessary construct, which procedures can be readily controlled (e.g., compared to induction of mutations using a chemical mutagenesis system).

These and other aspects, advantages, and features of the invention will become apparent to those persons skilled in the art upon reading the details of the invention as more fully described below.

### BRIEF DESCRIPTION OF THE DRAWINGS

FIG. 1 is a flow chart showing the general overview of the invention. Mice are infected with a recombinant retrovirus, which is integrated into the genome, any difference in phenotype is noted, and the DNA of the affected gene is subsequently isolated and sequenced to identify the point mutations.

FIG. 2 is a schematic diagram showing how the retrovirus, after integrating into the host DNA, provides for random integration of the Ig intronic enhancer element so that transcription of the element facilitates point mutations in a flanking host genomic sequence.

FIG. 3 is a schematic diagram showing the insertion of the hypermutation elements into a wild-type genome, and a non-comprehensive list of the kinds of retroviral constructs that can be used to insert the cis-acting hypermutation element into the cell or host of interest.

FIG. 4 is a schematic showing a wild type Akv1-99 virus and eight transgenic Akv viruses engineered to deliver a cis-acting hypermutation element. Solid region: R region of LTR; right-slanting diagonal lines: U5 region of LTR; vertical lines: viral structural genes; horizontal lines: U5 region of LTR (not including enhancer); left-slanting diagonal lines: native viral enhancer; cross-hatch: Mu intronic enhancer; dimples: kappa intronic enhancer; right-facing arrow: enhancer in forward orientation; left-facing arrow: enhancer in reverse orientation.

FIG. 5 is a schematic showing the sequence of a murine gene after infection with Akv1-99EkF and integration of hypermutation elements into the genome, as compared to the wild-type gene sequence.

FIG. 6 is a schematic showing the sequence of a murine gene after infection with Akv1-99EmuF and integration of hypermutation elements into the genome, as compared to the wild-type gene sequence.

FIG. 7 is a schematic showing the sequence of a murine gene after infection with Akv1-99EmuF and integration of hypermutation elements into the genome, as compared to the wild-type gene sequence.

FIG. 8 is a schematic showing the sequence of a murine gene after infection with Akv1-99EmuF and integration of hypermutation elements into the genome, as compared to the wild-type gene sequence.

### DETAILED DESCRIPTION OF THE INVENTION

Before the present invention is described, it is to be understood that this invention is not limited to particular embodiments described herein, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a vector” includes a plurality of such vectors and reference to “the host cell” or “host animal” includes reference to one or more host cells or host animals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

“Hypermutation” refers to a mechanism by which mutagenesis occurs at a rate approaching or exceeding that naturally occurring in the immunoglobulin variable region of the antibody heavy chain or antibody light chain genes, and in the flanking sequences; it is usually in the range of about 10⁻⁴ to 10⁻³ mutations/bp/generation, but can sometimes be an order of magnitude higher, or an order of magnitude lower.

A “point mutation” is a modification of a nucleic acid so that a single base pair is replaced with a different base pair (i.e., substituted) (or that a single nucleotides is replaced with a different nucleotide in the context of a single strand of nucleic acid). A mutated gene produced according to the invention can have multiple such point mutations, e.g., at least one, two, three, four, five, six, seven, eight, nine, ten, or more single nucleotide replacements (or base pair replacements in the context of a double-stranded nucleic acid).

A “small deletion” or “small insertion” as used herein is meant to refer to an insertion or deletion, respectively, of less than 10, usually, less than 5, more usually less than 3, generally 1, 2, or 3 base pairs (or nucleotides in the context of a single strand of nucleic acid). A mutated gene produced according to the invention can have multiple such small deletions and/or small insertions, e.g., at least one, two, three, four, five, six, seven, eight, nine, ten, or more single nucleotide replacements (or base pair replacements in the context of a double-stranded nucleic acid). A mutated gene produced according to the methods of the invention may also contain such small deletions, small insertions, or point mutations, and any combination or number of such mutations.

A “cis-acting hypermutation element” or “hypermutation element” refers to a nucleic acid that, when operably linked to another nucleic acid (e.g., particularly a heterologous nucleic acid, i.e., a nucleic acid other than that with which the cis-acting hypermutation element naturally occurs), provides for induction of mutations at a rate orders of magnitude higher than the normal spontaneous mutation rate within the flanking sequence (e.g., within the heterologous flanking sequence). Cis-acting hypermutation elements include any sequence that acts to facilitate introduction of a point mutation, small insertion, and/or small deletion in an adjacent genomic sequence (particularly an adjacent heterologous genomic sequence), either directly or indirectly. Examples of such cis-acting hypermutation elements include the immunoglobulin intronic enhancers, such as the heavy chain large intronic enhancer and the kappa intronic enhancer. Such “cis-acting hypermutation elements” as used in the present invention are generally provided in a vector (e.g., a viral vector or DNA construct) for insertion adjacent into a host cell genome so as to provide for hypermutation of an endogenous host gene adjacent the inserted hypermutation element. In one embodiment, the hypermutation element is inserted adjacent a host gene other than an immunoglobulin gene.

The terms “immunoglobulin intronic enhancer” and “immunoglobulin hypermutation element” are used interchangeably herein to refer to the enhancer element that is associated with hypermutation in an immunoglobulin variable (V) region of a heavy chain- or light chain-encoding gene, as well as subfragments having such hypermutation activity.

The terms “heavy chain large intronic enhancer element,” “heavy chain large intronic enhancer fragment,” and “heavy chain large intronic enhancer” are used interchangeably herein to refer to an enhancer element of an heavy chain immunoglobulin locus, exemplified by the enhancer contained within the XbaI-XbaI fragment described in Bachl et al., supra, as well as one or more subfragments of the XbaI-Xba-I fragment which retain hypermutation activity.

The terms “kappa (K) intronic enhancer fragment”, “kappa (K) enhancer element”, and “kappa (K) enhancer” are used interchangeably herein to refer to an enhancer of a kappa light chain immunoglobulin locus, exemplified by the enhancer described in Max et al. (The nucleotide sequence of a 5.5-kilobase DNA segment containing the mouse kappa immunoglobulin J and C region genes. J. Biol. Chem. 256 (10), 5116-5120 (1981)), and as defined in Klix et al. (Multiple sequences from downstream of the J Kappa cluster can combine to recruit somatic hypermutation to a heterologous, upstream mutation domain. Eur. J. Immunol. 1998. 28: 317-326), and can be one or more subfragments thereof which retain hypermutation activity.

A “hypermutation-inducing vector” or “hypermutation-inducing construct”, as used interchangeably herein, is a vector minimally comprising a cis-acting hypermutation element, which element, upon its insertion adjacent to a sequence of a gene, facilitates introduction of point mutations in that gene. In one embodiment, the hypermutation element is an immunoglobulin intronic enhancer, such as the heavy chain large intronic enhancer element or the kappa intronic enhancer. The hypermutation element can be used in the genomic 5′ to 3′ orientation and can also be used in the 3′ to 5′ reverse orientation, although this latter embodiment is less preferred in the case of the heavy chain large intronic enhancer element.

“Trans-acting hypermutation factor(s)” refers to a factor(s) that acts in trans to the cis-acting hypermutation element to effect introduction of mutations at a higher than normal rate in a sequence adjacent the cis-acting element.

A “trans-acting hypermutation factor(s) positive cell” or “mutator positive cell” is a cell or cell line having cellular factors sufficient to work in combination with enhancers to effect hypermutation, e.g., has the trans-acting factors that, in combination with the cis-acting hypermutation elements, cause hypermutation. The cells or cell line can be of pre-B lymphocyte origin (e.g., such as the 18-81 cell line), or B cell-origin, or can be any other cell or cell line transfected with factors determined to effect hypermutation.

“Essentially evenly distributed mutagenesis”, “substantially evenly distributed mutagenesis”, “random mutagenesis” and like terms are used herein to refer to the nature of the mutations generated in a gene, i.e., that mutagenesis is other than directed to a specific, pre-determined candidate gene. Mutagenesis of this nature encompasses mutagenesis that may occur in “hot spots” due to sequence variability in the genome.

“Gene” as used herein is meant to refer to a nucleic acid comprising, at minimum, a sequence encoding a promoter and a coding sequence (e.g., in mammalian genomes, at least one exon), wherein the promoter and coding sequence are operably linked. In general, “gene” as used herein is meant to refer to a nucleic acid sequence that is expressed by transcription from the operably linked promoter to provide for production of a gene product from the associated coding sequence. Furthermore, “genes” generally refers to a nucleic acid having at least one sequence susceptible to hypermutation following operative insertion of a cis-acting hypermutation element according to the invention.

“Endogenous gene” as used herein means a nucleic acid in a genome of a host cell that is present in its naturally-occurring position in the genome.

The terms “nucleic acid” and “polynucleotide” refer to deoxyribonucleotides (e.g., DNA and cDNA) or ribonucleotides (e.g., mRNA) and polymers thereof in either single- or double-stranded form and may be naturally-occurring, synthetically produced, or recombinant. Unless specifically limited, the term encompasses nucleic acids containing analogues of natural nucleotides that have similar binding properties as the reference nucleic acid and are metabolized in a manner similar to naturally occurring nucleotides. Unless otherwise indicated, reference to a particular nucleic acid sequence is meant to encompass conservatively modified variants thereof (e.g., degenerate codon substitutions) and complementary sequences in addition to the specific sequence explicitly indicated. Specifically, degenerate codon substitutions may be achieved by generating sequences in which the third position of one or more selected (or all) codons is substituted with mixed-base and/or deoxyinosine residues. Unless otherwise indicated, a particular nucleic acid sequence includes the perfect complementary sequence thereof.

The phrase “a nucleic acid sequence encoding” or “a polynucleotide encoding” refers to a nucleic acid that contains sequence information for an mRNA, a structural RNA such as rRNA, a tRNA, or a binding site for a trans-acting regulatory agent. This phrase specifically encompasses degenerate variants of the native sequence (i.e., different codons which encode a single amino acid) or sequences that may be introduced to conform with codon preference in a specific host cell.

By “construct” is meant a recombinant nucleic acid, generally recombinant DNA, that has been generated for the purpose of manipulating nucleotide sequence(s), e.g., in the construction of other recombinant nucleotide sequences, for introduction into the genome of a host cell (e.g., as a integrated or episomal element), and the like. “Constructs” includes constructs having nucleic acid of any origin (e.g., viral, bacterial, eukaryotic (e.g., mammalian), and other origins.

By “viral vector” or “viral construct” is meant a construct that contains nucleic acid of viral origin, and which can be packaged in a viral particle to accomplish infection and transfection of a target cell with a nucleotide sequence of interest (i.e., transduction).

By “operably linked” or “operatively inserted” is meant that a DNA sequence and a regulatory sequence(s) (e.g., a promoter) are connected in such a way as to permit gene expression when the appropriate molecules (e.g., transcriptional activator proteins) are bound to the regulatory sequence(s). Operably linked can also refer to the joining of cis-acting elements (e.g., hypermutation elements). “Operably linked” as used in the context of a cis-acting hypermutation element operably linked to a nucleic acid is meant to indicate that the cis-acting hypermutation element is associated with the adjacent nucleic acid so that transcription of the hypermutation element facilitates introduction of one or more mutations in the adjacent nucleic acid.

“Substantial identity,” when referring to the polynucleotides of this invention, means polynucleotides having at least 80%, typically at least 90% and preferably at least 95% sequence identity to a nucleotide sequence of interest. Sequence identity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990) J. Mol. Biol. 215:403-410. Percent identity can be determined using the BLASTN program with the default settings (including default gap weights) with the DUST filter selected.

Nucleic acids having sequence similarity can also be detected by hybridization under low stringency conditions, for example, at 50° C. and 6×SSC (0.9 M sodium chloride/0.09 M sodium citrate) and remain bound when subjected to washing at 55° C. in 1×SSC (15 mM sodium chloride/1.5 mM Na citrate). Sequence identity may be determined by hybridization under stringent conditions, for example, at 50° C. or higher and 0.1×SSC (15 mM sodium chloride/0.15 mM sodium citrate). By using probes, particularly labeled probes of DNA sequences, one can isolate orthologous, homologous or related genes. The source of orthologous or homologous genes may be any species, e.g. primate species, particularly human; rodents, such as rats and mice, canines, felines, bovines, ovines, equines, yeast, Drosophila, Caenhorabditis, etc.

By “transgenic animal” is meant a non-human animal, usually a mammal (e.g., mouse, rat, rabbit, hamster, guinea pig, pig, cow, sheep, goat, monkey, etc.), having a non-endogenous (i.e., heterologous) nucleic acid sequence present as an extrachromosomal element in a portion of its cells or stably integrated into its genomic DNA. Generally as used herein, “transgene” refers to a stably integrated transgene unless otherwise specifically noted. Heterologous nucleic acid can be introduced into the germ line of such transgenic animals by genetic manipulation of, for example, zygote, embryos or embryonic stem cells of the host animal. Alternatively, and generally preferably in the context of the present invention, an animal can be made transgenic by introduction of the transgenic element by, for example, infection with a integrating construct or integrating vector (e.g., infection with an integrating viral vector, such as a retroviral vector).

The term “transgene” is used herein to describe genetic material which has been or is adapted for being artificially inserted into the genome of a non-human, living vertebrate animal, particularly a mammal.

By “heterologous” is meant that, for example, two elements in consideration are derived from different genes, and thus each element is flanked by one or more sequences with which the element is not normally found in nature. “Heterologous” can also indicate that the elements are derived from different sources, e.g., different species. Thus, for example, a hypermutation element that is heterologous to a gene endogenous to a host cell means that the hypermutation element is adjacent a gene with which it is not normally found in nature.

By “recombinant” as used in the context of nucleic is meant that the nucleic acid contains an alteration that is not normally found in nature, e.g., as a result of genetic manipulation techniques.

Overview of the Invention

The invention is based on the development of a system to provide for random and essentially evenly distributed mutagenesis in flanking genomic sequences adjacent the site of integrate in a manner that allows for ready identification of the adjacent gene that was mutated. This high-rate mutagenesis system utilizes a vehicle, such as a recombinant virus, comprising a hypermutation element. In general, the invention involves genomic integration of a cis-acting hypermutation element adjacent to a candidate gene of a host cell. Genomic integration of the hypermutation element is preferably accomplished using a replicating delivery vehicle, such as a replication competent retroviral vector. Preferably, integration is essentially random so as to provide for analysis of a variety of candidate genes in the host genome.

Hypermutation occurs in those cells 1) in which the cis-acting element is inserted so that the cis-acting element is transcribed when the adjacent coding sequence is transcribed, and 2) having, or modified to have, the appropriate trans-acting factors that act in conjunction with the integrated cis-acting element to effect hypermutation of the adjacent nucleic acid. In general, hypermutated cis-sequences present on the vector recruit trans-acting elements, resulting in saturation mutagenesis of a gene 5′ of the hypermutation element at a rate of about 10⁻³ to 10⁻⁴/bp/generation. Mutations generated in the host genome are thus essentially random both with respect to the gene mutated and the nature of the mutation introduced. Furthermore, the mutations introduced by the hypermutation element are point mutation, small insertions and small deletions, thus allowing for analysis of the effect of small changes (e.g., of 1 2, or 3 base pairs within a single site) upon the oncogenic potential of the affected gene.

The invention will now be described in more detail.

Hypermutation Inducing Constructs

Any of a variety of constructs can be used in connection with the present invention. In general, the vector of the invention provides for integration of a cis-acting hypermutation element into a genome of a host cell so as to facilitate hypermutation of a transcribed sequence adjacent, and generally 5′ of, the integrated cis-acting hypermutation element. This can be accomplished by integration of all or a part of the construct into the genome. Preferably, the construct is replicating within the animal, e.g., where the construct is a viral vector, the virus is replication competent. In isolated cells or cell lines, the virus can be replication deficient.

The hypermutation inducing constructs of the invention minimally comprise a hypermutation element (e.g., an immunoglobulin intronic enhancer having hypermutation activity, e.g., of the heavy chain or light chain intronic enhancer from an immunoglobulin locus) and one or more element to facilitate integration of the construct into the genome of a host cell (i.e., the constructs are adapted for integration into the genome of a host cell). Additional elements can include a transcriptional enhancer element, a replication of origin (e.g., to facilitate production of the vector), and the like.

Exemplary constructs of interest include, but are not necessarily limited to retroviral vectors (e.g., MMTV, MLV, and the like); but they can also include plasmids, cosmids, and other viral vectors, including, for example, lentiviral vectors, herpes virus vectors (e.g., HSV), adenoviral vectors, and adeno-associated viral (AAV) vectors. Other exemplary constructs useful in delivering the cis-acting element to the genome of a host cell include transposon or positive or negative selection cassettes.

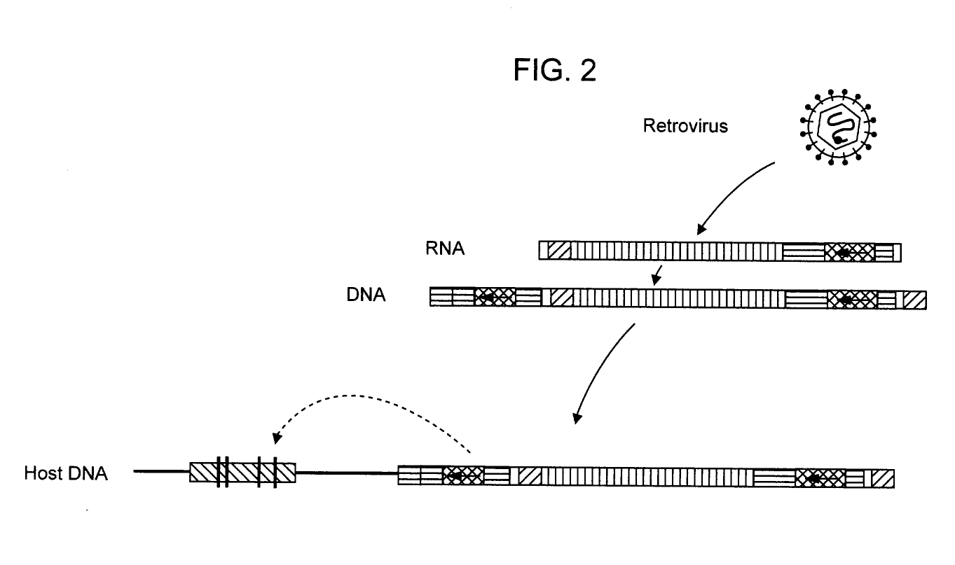

The hypermutation-inducing constructs of the invention are adapted for integration into the genome of a host cell, usually a vertebrate cell, particularly a mammalian cell. By “adapted for integration into a host cell genome” is meant that the vector contains one or more nucleic acid elements that promote introduction of at least the cis-acting hypermutation element into a host cell genome, e.g., by homologous recombination or viral-mediated integration. Preferably, integration of the hypermutation element is essentially random, e.g., by having a viral vector particularly a retroviral vector as the basis for the hypermutation-inducing construct. In general, integration of the hypermutation-inducing construct, or at least the cis-acting hypermutation element of the construct, facilitates hypermutation of the flanking genomic sequences when the sequences are transcribed. In one embodiment, integration of a cis-acting hypermutation element at a position that is 3′ (downstream) of a genomic sequence (e.g., a genomic coding sequence) provides for introduction of mutations (e.g, point mutations, small deletions, small insertions) in the genomic sequence, and particularly in the sequence 5′ to the site of integration (see, e.g., FIG. 2).

A feature of the invention is that the hypermutation-inducing constructs of the invention are adapted for introduction into a host cell genome, so as to facilitate introduction of one or more mutations (e.g., point mutations, small deletions, small insertions) into a genomic sequence endogenous to the host cell (e.g., a gene that is normally present in the host cell genome, and is mutated at its naturally-occurring position within the genome, i.e., in situ). The hypermutation-inducing constructs of the invention themselves need not contain, and may preferably not contain, a nucleic acid such as a gene product-encoding sequence, into which mutations are to be introduced, since the nucleic acid of interest for hypermutation is one present in the host cell genome. In addition, the hypermutation construct need not contain a promoter that will provide for transcription of the hypermutation element and an adjacent nucleic acid encoding a sequence that is to be hypermutated. Instead, genomic integration of the hypermutation element can result in transcription of the hypermutation element being driven by a promoter element of a genomic sequence, e.g., a promoter element of a gene endogenous to the host cell genome into this which the hypermutation element is integrated. Thus, in one embodiment of particular interest, the hypermutation-inducing construct does not contain a nucleic acid encoding a gene product (e.g., a polypeptide, such as a reporter polypeptide or an immunoglobulin polypeptide) operably linked to the hypermutation element, and further need not contain a promoter to drive transcription of the hypermutation element.

Hypermutation Enhancer Element

Generally speaking, the invention exploits elements of the immunoglobulin (Ig) hypermutation system to introduce mutations into heterologous target genes in situ. The invention is exemplified by elements of the murine Ig hypermutation system. The invention, however, is not limited to the use of murine components. For example, human, rat, rabbit, chicken, hamster, monkey components can also be used. In addition, components of analogous Ig hypermutation systems can also be used. It may be preferable to use hypermutation elements that are derived from an Ig locus of the same species as that of the host cell in which hypermutation is desired. Reference to murine components throughout is not meant to be limiting, but rather is for clarity and convenience's sake only.

The constructs of the invention minimally comprise at least one cis-acting hypermutation element, usually an immunoglobulin intronic hypermutation element. In some embodiments, the immunoglobulin intronic enhancer is a heavy chain large intronic enhancer or a kappa (light chain) intronic enhancer. In other embodiments, the hypermutation element may comprise multiple hypermutation elements, e.g., two or more hypermutation elements, which hypermutation elements may be the same or different, with the proviso that, when operably linked to a genomic nucleic acid, the hypermutation elements facilitate mutation of the genomic flanking sequence. In some embodiments, the hypermutation element may be in the reverse or forward orientation, where the forward orientation may be of particular interest. In some embodiments, it may be desirable to provide the hypermutation element as a minimal sequence that facilitates hypermutation of an adjacent, operably linked nucleic acid.

Where the cis-acting hypermutation element comprises an immunoglobulin intronic enhancer, integration events that result in induction of hypermutation of the adjacent gene will generally be those that result in insertion of the immunoglobulin enhancer so that the 5′ end is up to 3 kb 3′ of the 3′ end of the target gene (i.e., the gene adjacent the integrated enhancer), preferably less than about 2 kb, more preferably less than about 1 kb from the 3′ end of the target gene. Function of the enhancer is optimal when the 5′ end of the enhancer is not greater than about 3 kb from the 3′ end of the coding sequence in which mutation is desired. Hypermutation can occur, but is less efficient, when the immunoglobulin enhancer is positioned greater than 3 kb 3′ of the target gene.

In some embodiments, the cis-acting hypermutation element is provided in a hypermutation construct with a “non-specific” transcriptional enhancer, i.e., an enhancer that enhances the overall transcription level, and does not by itself provide for detectable or significant hypermutation. In some embodiments, the transcriptional enhancer is generally present in the construct at a distance greater than 1 kb from the cis-acting hypermutation element. In some embodiments, the transcriptional enhancer is an enhancer of a viral construct, e.g., an enhancer located in a long terminal repeat (LTR) of a viral-based construct.

In a preferred embodiment, the cis-acting hypermutation element(s) are present in the construct so that integration results in insertion of the elements in the orientation in which they are naturally found in the genome. The enhancer sequences present in the genomic immunoglobulin gene are present in a “genomic orientation.” Flipping of the cis-acting hypermutation element so that each sequence is in a 3′ to 5′ orientation (as opposed to the 5′ to 3′ orientation in the native genomic configuration), represents the “reverse orientation.” The cis-acting hypermutation element can also be present in reverse orientation, but this is less preferred.

A preferred heavy chain large intronic enhancer is the heavy chain large intronic enhancer contained within the XbaI-XbaI fragment, approximately 1 kb in size, of the mouse heavy chain locus. An exemplary mu intronic enhancer is one having the nucleotide sequence:

| | |
|---|---|
| ctagagaggt ctggtggagc ctgcaaaagt ccagctttca aaggaacaca gaagtatgtg tatggaatat tagaagatgt | (SEQ ID NO:1) |
| tgcttttact cttaagttgg ttcctaggaa aaatagttaa atactgtgac tttaaaatgt gagagggttt tcaagtactc | |
| atttttttaa atgtccaaaa tttttgtcaa tcaatttgag gtcttgtttg tgtagaactg acattactta aagtttaacc | |
| gaggaatggg agtgaggctc tctcataccc tattcagaac tgacttttaa caataataaa ttaagtttaa aatattttta | |
| aatgaattga gcaatgttga gttgagtcaa gatggccgat cagaaccgga acacctgcag cagctggcag | |
| gaagcaggtc atgtggcaag gctatttggg gaagggaaaa taaaaccact aggtaaactt gtagctgtgg | |
| tttgaagaag tggttttgaa acactctgtc cagccccacc aaaccgaaag tccaggctga gcaaaacacc | |
| acctgggtaa tttgcatttc taaaataagt tgaggattca gccgaaactg gagaggtcct cttttaactt attgagttca | |
| accttttaat tttagcttga gtagttctag tttccccaaa cttaagttta tcgacttcta aaatgtattt agaattcatt | |
| ttcaaaatta ggttatgtaa gaaattgaag gactttagtg tctttaattt ctaatatatt tagaaaactt cttaaaatta | |
| ctctattatt cttccctctg attattggtc tccattcaat tattttccaa tacccgaagt ctttacagtg actttgttca | |
| tgatcttttt tagttgtttg ttttgcctta ctattaagac tttgacattc tggtcaaaac ggcttcacaa atctttttca | |
| agaccacttt ctgagtattc attttaggag aaatattttt tttttaaatg aatgcaatta t; | |

wherein all or a hypermutation promoting portion of the sequence may be used.

Where the hypermutation element is a kappa intronic enhancer, the following kappa intronic enhancer element, or a hypermutation-promoting portion thereof, may be preferred:

| | |
|---|---|
| agcttaatgtatataatcttttagaggtaaaatctacagccagcaaaagtcatggtaaatattctttgactgaactctcact | (SEQ ID NO:2) |
| aaactcctctaaattatatgtcatattaactggttaaattaatataaatttgtgacatgaccttaactggttaggtaggatatttttctt | |
| catgcaaaaatatgactaataataatttagcacaaaaatatttcccaatactttaattctgtgatagaaaaatgtttaactcagctacta | |
| taatcccataattttgaaaactatttattagcttttgtgtttgacccttccctgccaaaggcaactatttaaggaccctttaaaactctt | |
| gaaactactttagagtcattaagttatttaaccacttttaattactttaaaatgatgtcaattcccttttaactattaatttattttaag | |
| gggggaaaggctgctcataattctattgtttttcttggtaaagaactctcagtttctgttttactacctctgtcacccaagagttggcat | |
| ctcaacagaggggactttccgagagccatctggcagttgcttaagatcagaagtgaagtctgccagttcctcctaggcaggtggcccaga | |
| ttacagttgacctgttctggtgtggctaaaaattgtcccatgtggttacaaaccattagaccagggtctgatgaattgctcagaatattt | |
| ctggacacc. | |

Another embodiment utilizes hypermutation-inducing fragments of one or both enhancers. Hypermutation-inducing fragments can be identified in a number of ways. One way is to perform deletion analysis by constructing hypermutation cassettes containing various enhancer deletion mutants and a reporter gene. The hypermutation efficacy of the enhancer deletion mutant can be assessed by determining the rate of mutation of the reporter gene (see, e.g., U.S. Pat. No. 5,885,827). Deletion mutants can be prepared in a variety of ways. Oligonucleotides can be designed containing fragment sequences to be tested. Alternatively, a more random approach is to linearize the expression vector by restriction digest within an enhancer, followed by subsequent exonuclease treatment and religation. Yet another method is to simply use restriction digests to remove sections of DNA.

Additional Elements of the Construct

The construct can also include other elements, e.g., to facilitate production or handling of the construct, etc. Exemplary additional elements include, but are not necessarily limited to, sequences permitting replication of the cassette in eukaryotes, or prokaryotes, or both (e.g., shuttle vectors), and selectable markers for both prokaryotic and eukaryotic systems (e.g., Neo®).

Hosts and Host Cells

Host cells contemplated for use in the invention include any eukaryotic host cell, usually a vertebrate host cell, more usually a mammalian host cell, which cell may be a cultured cell or cell line, or which cell may be present in a non-human animal.

In general, the host cell may be any suitable cell of a eukaryotic host, into the genome of which the cis-acting hypermutation element can be genomically integrated, e.g., host cells that are susceptible to infection by a recombinant viral vector comprising the cis-acting hypermutation element. Hypermutation will occur in mutator positive cells present in the host or in the culture.

The host cell can be any suitable eukaryotic cell, usually a vertebrate cell, such as a primate (e.g., human, monkey, ape, chimpanzee, etc.), rodent (e.g., mouse, rat, hamster, etc.), guinea pig, lagomorph (e.g., rabbit, etc.), avian (e.g., chicken, turkey, etc.), reptile, amphibian (e.g., frog), fish, ungulate (e.g., cow, pig, sheep, etc.), cat, dog, or other easily manipulated organism. Where the hypermutation is carried out in an organism, the host is generally a non-human animal. A non-human animal having a genomically integrated cis-acting hypermutation element according to the invention is considered to be transgenic for the hypermutation element.

As noted above, a “trans-acting hypermutation factor(s) positive cell” or “mutator positive cell” is a cell having cellular factors that work in combination with enhancers to effectuate hypermutation. The cell in which hypermutation is to occur is preferably of B lymphocyte origin. Alternatively, the host cell can be modified to express or contain trans-acting factors that facilitate hypermutation in conjunction with the cis-acting hypermutation element, for example, any cell into which the gene encoding Activation-induced Cytidine Deaminase (AID) (see Yoshikawa et al., AID enzyme-induced hypermutation in an actively transcribed gene in fibroblasts. Science. Jun 14, 2002;296(5575):2033-6). has been introduced.

Cells that naturally express functional trans-acting hypermutation factors include Abelson virus-transformed pre-B cells and activated B lymphocytes, as well as other cells (including cell lines) derived from B lymphocytes.

One way to produce a cell transfected with a factor required for hypermutation is to construct a cDNA library in a hypermutation-inducing vector containing a reporter gene. The cDNA can be prepared by conventional techniques from a mutator-positive cell line. The cDNA-reporter library construct is then transfected into a mutator-negative cell line, hypermutation is allowed to proceed as the transfectant cells are grown to a desired density, and the resultant pool is screened for a mutant phenotype. Alternatively, a vector pool containing the cDNA library can be co-transfected with the hypermutation reporter vector. Another way to make a mutator-positive cell line is to narrow the cDNAs tested to those encoding proteins known to bind E-boxes within various enhancers. It is possible that more than one factor might be required for hypermutation activity, and consequently, initial mutation rates can be expected to be less than 10⁻⁴/bp/generation, but should be greater than 10⁻⁷/bp/generation. It is also possible that some factors may be composed of polypeptide subunits and expression cloning procedures could be modified to achieve complementation. Preferably, the protein AID is present.

Delivery Vehicles and Methods of Delivery

Any of a variety of gene delivery methods can be used to deliver the cis-acting hypermutation element to the genome of a host cell. In a preferred embodiment, the enhancer is present on a non-oncogenic, replicating vector which facilitates genomic insertion of the enhancer, preferably in an essentially random manner.

In one embodiment, the host is a mouse and the construct is a retroviral vector derived from MMTV (Mouse Mammary Tumor Virus) or MLV (Murine Leukemia Virus), such as Akv, or any other retrovirus that infects B lymphocytes. After producing a sufficient amount of the virus, it is then administered to the animal to infect the host. This can be done by any suitable route, such as intraperitoneal, intravenous, and the like. Preferably the retrovirus should continue to spread after the initial infection, i.e., the retroviral vector is preferably replication competent.

In cases where viral replication is not desired, a replication defective retroviral vector can be used. In this case, efficient gene transduction and integration generally requires the presence of 4 cis-acting elements in the retroviral vector: (i) a promoter and a polyadenylation signal; (ii) a packaging signal to direct incorporation of vector RNA into virion; (iii) a primer-binding site and polypurine tract for initiation and R region for strand transfer during reverse transcription; and (iv) sequences at the termini of the viral LTR for integration. Other than the cis-acting elements, all of the coding regions of a retrovirus can be removed. A common design involves the replacement of retroviral sequences with a transgene to create replication-defective vectors. The vector by itself is therefore incapable of making viral proteins required for additional rounds of infection. Viral proteins needed for the initial infection can be provided in trans by a helper virus or, more commonly, by a retroviral packaging cell line. The packaging cell is designed to provide all viral proteins but not to package nor transmit the RNAs encoding these functions. Retroviral vectors produced by packaging cells can transduce cells but cannot replicate further.

Cells that are infected by the introduced virus incorporate the viral construct that includes the Ig enhancer into their genome. Hypermutation occurs in the cells 1) in which the cis-acting hypermutation element is integrated and transcribed, and 2) that produce, or have been modified so as to produce, the necessary trans-acting factors which work in conjunction with the genomically integrated enhancer. Such cells include, but are not limited to, B lymphocytes, particularly activated B lymphocytes, and cells derived from such cells.

After allowing for infection and integration, the animal is subsequently observed for tumor formation according to methods known in the art. For example, detection of B-cell or other tumors can be accomplished by observing the host for protruding tumorous growths or lump formation. B-cell tumors can also be associated with an enlarged spleen. B-cell tumors can be typed by analyzing cell surface markers, or by nucleic acid amplification of a portion of the immunoglobulin locus.

Nucleic Acid Delivery Systems

A variety of delivery systems or vehicles can be used to accomplish introduction of a construct of the invention into the genome of a host cell to effect random mutagenesis. Preferably, the construct is delivered using a viral vector that effects, as near as possible, essentially evenly distributed, random genomic integrations.

Additional delivery vehicles are described below.

Viral Delivery Systems. Viral delivery systems are the preferred means of introducing a construct of the invention into the genome of the cell. Viral vectors suitable for use in the present invention include any viral vector suitable for delivery of cis-acting element into the genome of a host cell. Viral vectors can be either replication-competent or replication deficient, whereas the replication competent viruses are preferred. Viral vectors of particular interest include, but are not necessarily limited to, those vectors based on retroviruses (including pseudotyped retroviruses, and lentiviruses, such as HIV-based vectors, which may not require cell division), Sindbis virus, adenovirus, adeno-associated virus (AAV), adenovirus, Rous sarcoma virus (RSV), poxvirus, semliki-forest virus (SFV), MLV, and herpesvirus (e.g., CMV, HSV, etc.). Exemplary viral vectors suitable for use in the in vivo delivery methods of the invention are described below.

Retroviral vectors. Retroviral vectors are particularly useful in certain applications. The design of replication competent or replication defective retroviral vectors is well known to one of skill in the art. Preparation of retroviral vectors and their uses are described in many publications including EP-A 0 178 220, U.S. Pat. No. 4,405,712; Gilboa (1986) Biotechniques 4:504-512; Mann et al. (1983) Cell 33:153-159; Cone et al. (1984) Proc. Natl. Acad. Sci. U.S.A. 81:6349-6353; Eglitis et al. (1988) Biotechniques 6:608-614; Miller et al. (1989) Biotechniques 7:981-990; WO 92/07943; Solaiman et al. Mol Reprod Dev June 2000;56(2 Suppl):309-15; and Bell et al. Mol Biotechnol June 1997;7(3):289-98.

The retroviral vector particles are prepared by recombinantly inserting a nucleic acid encoding a cis-acting hypermutation element of interest into a retrovirus vector and, packaging the vector into retroviral vector particles. Where the retroviral vector is to be replication incompetent, the vector is packaged using retroviral capsid proteins expressed in a packaging cell line. The resultant retroviral vector particle is capable of integrating into the host cell genome as a proviral sequence containing the nucleic acid of interest.

Packaging cell lines are generally used to prepare the retroviral vector particles. A packaging cell line is a genetically constructed mammalian tissue culture cell line that, where replication-defective viruses are to be made, produces the necessary viral structural proteins required for packaging and which are not functional in the retroviral genome.

A number of packaging cell lines are available. Examples of packaging cells lines include Crip, GPE86, PA317 and PG13 (see, e.g., Miller et al. (1991) J Virol. 65:2220-2224). Examples of other packaging cell lines are described in Cone et al. (1984) Proc. Natl. Acad. Sci. U.S.A. 81:6349-6353; Danos et al. (1988) Proc. Natl. Acad. Sci. U.S.A. 85:6460-6464; Eglitis et al. (1988) Biotechniques 6:608-614; Miller et al. (1989) Biotechniques 7:981-990.

Amphotropic or xenotropic envelope proteins, such as those produced by PA317 and GPX packaging cell lines may also be used to package the retroviral vectors. Also of interest is the use of packaging cell lines that produce pseudotyped viral particles having vesicular stomatitis viral coat protein (VSV G), which may be obtained either by transient transfection of a packaging cell line (e.g., a cell expressing a retroviral gag and pol) or through use of a packaging cell line that stably expresses VSV G.

Adeno-Associated Virus (AAV) Vectors. Because of their demonstrated ease of use, broad host range, stable transmission to daughter cells, high titer/microgram DNA, and stable integration, (Lebkowski et al. (1988) Mol. Cell. Biol. 8:3988-3996), AAV vectors may be useful in delivery of hypermutation-inducing elements to host cells (see, e.g., Goeddel (ed.) (1990) Meth. Enzymol. 185, Academic Press, Inc., San Diego, Calif.; Krieger, (1990) in: Gene Transfer and Expression—A Laboratory Manual, Stockton Press, New York, N.Y.). AAV generally requires helper viruses such as adenovirus or herpes virus to achieve productive infection.

In the absence of helper virus functions, AAV integrates into a host cell's genome. The integrated AAV genome alone has no detectable pathogenic effect. The integration step allows the AAV genome to remain genetically intact until the host is exposed to the appropriate environmental conditions (e.g., a lytic helper virus), whereupon it re-enters the lytic life-cycle. For discussion of AAV, AAV vectors, and uses thereof, see, e.g., Samulski (1993) Curr. Op. Genet. Dev. 3:74-80 (references cited therein for an overview of the AAV life cycle); West et al. (1987) Virol. 160:3847; U.S. Pat. No. 4,797,368; WO 93/24641; Kotin (1994) Hum. Gene Ther. 5:793-801; Muzyczka (1994) J. Clin. Invest. 94:1351; and Samulski, supra.

AAV displays a very broad range of hosts including chicken, rodent, monkey and human cells (Muzycka, N. (1992) Curr. Top. Microbiol. Immunol. 158:97-129; Tratschin et al. (1985) Mol. Cell. Biol. 5:3251-3260; and Lebkowski et al. (1988) Mol. Cell. Biol. 8:3988-3996). They efficiently transduce a wide variety of dividing and non-dividing cell types in vitro (Flotte et al. (1992) Am. J. Respir. Cell. Mol. Biol. 7:349-356; Podsakoff et al. (1994) J. Virol. 68 5655-5666; and Alexander et al (1994) J. Virol. 68:8282-8287). AAV vectors have been demonstrated to successfully transduce hematopoietic progenitor cells of rodent or human origin (Nahreini et al. (1991) Blood 78:2079). It is believed that AAV may infect virtually any mammalian cell type. Production of AAV vectors can be accomplished according to methods well known in the art.

Non-viral Delivery Systems

While viral delivery systems are preferred in some embodiments it may be desired to use a non-viral means of effecting introduction of a construct of the invention into a host cell. Non-viral delivery systems include naked nucleic acid (see, U.S. Pat. Nos. 5,693,622 and 5,580,859), transfection-facilitating proteins (e.g., DNA-protein formulations), liposomal formulations (see, U.S. Pat. Nos. 4,394,438; and 5,459,127), charged lipids, calcium phosphate precipitating agents, DNA-targeting ligand formulations (e.g., to facilitate receptor-mediated endocytosis), polycationic substances such as poly-L-lysine or DEAC-dextran (Felgner et al. (1987) Proc. Natl. Acad. Sci. U.S.A. 84:7413-7417), and the like. Methods and compositions for production of such non-viral delivery systems are well known in the art

Tagging and Identification of Hypermutated Genes

Once a phenotype, such as tumor formation, has been observed, the gene having the mutated sequence that is associated with tumor formation, e.g., the gene that has become oncogenic as a result of the mutations introduced by hypermutation, is identified and, optionally, isolated for further analysis.

In general, the cell having the phenotype of interest (e.g., a tumor cell) is isolated from the host, and nucleic acid isolated from the cell. In one embodiment, the host cells are B cells. In one embodiment, the tissue is first processed to produce single cell suspensions, and nucleic acid isolated from the single cells.

The genomic sequence that has undergone hypermutation is identified using the inserted hypermutation element as a genetic tag. In one embodiment, primers are designed based upon a sequence of the inserted hypermutation construct or, more particularly, a sequence of an inserted cis-acting hypermutation element, and the nucleic acid amplified by anchored PCR; or circularization of restriction-digested DNA with subsequent PCR using a construct-derived primer (e.g., a viral primer where the construct is a viral construct), one of which may be the Ig enhancer—so as to accomplish amplification of the hypermutated gene adjacent the inserted hypermutation construct or the inserted cis-acting hypermutation element. The resulting PCR product is sequenced, and these sequences used to facilitate identification of the hypermutated gene. For example, the sequences can be used to search various public or commercially available sequence databases to identify homologous sequences. Alternatively, or in addition, the amplified sequences can be used to isolate nucleic acid encoding the hypermutated or naturally-occurring (e.g., the sequence prior to hypermutation) coding sequence.

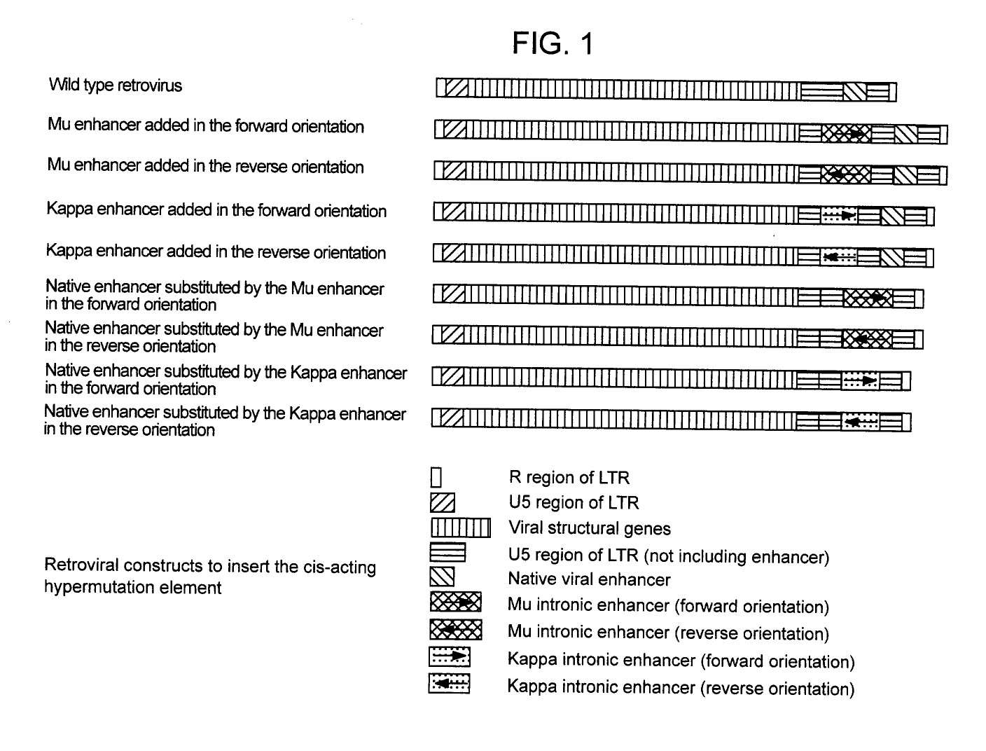

As shown in an example of the invention illustrated in FIG. 1, using the tumorigenic model, newborn mice are infected with a recombinant retrovirus that contains the cis-acting hypermutation element. The hypermutation element, once integrated into the genome, generates point mutations, as illustrated schematically in FIG. 2. Some of the mutations will lead to tumor formation in the mice. The tumor phenotype is identified and the DNA of the mouse is then sequenced to determine the location of the point mutation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Tumorigenic Point Mutations

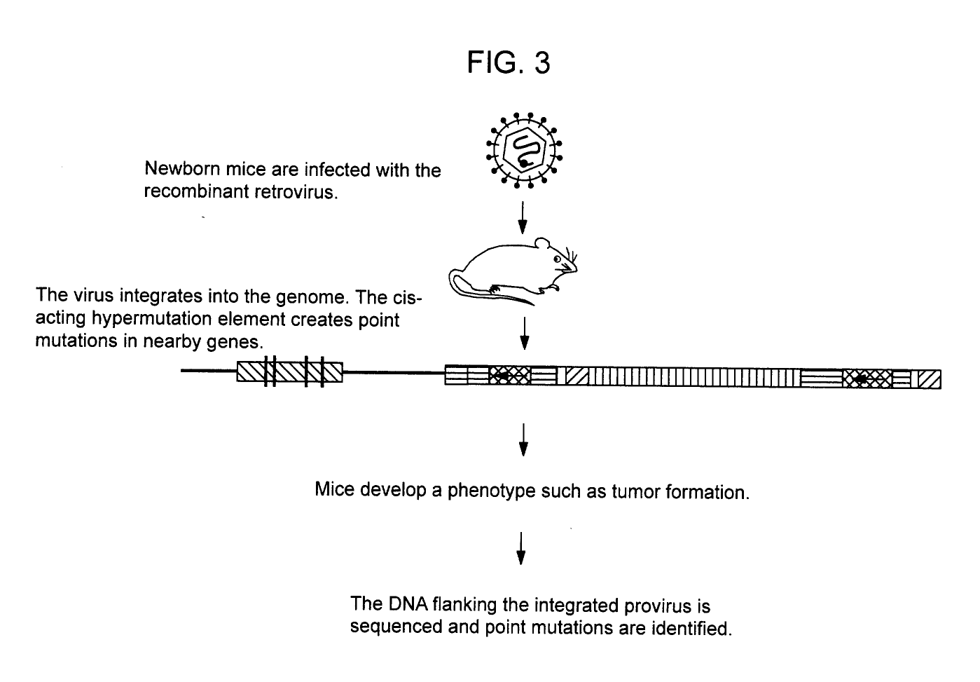

A mu enhancer (SEQ ID NO:1), or a cis-acting hypermutation element, as shown in FIG. 3, contained within an XbaI-XbaI fragment is inserted into a vector in the forward direction using protocols well known to one of skill in the art. The vector also has a retroviral gag, pol and env protein encoding sequences. The vector exemplified here is an MLV vector, although any suitable retrovirus can be used.

The construct is integrated into the genome of a mouse (preferably a newborn mouse, so that there is no immune response to the virus) following intraperitoneal (i.p.) injection. Following infection, the virus replicates into dsDNA and inserts into the host genome. Alternatively the vector can be delivered subcutaneously (SC) by injecting 1×10⁷ particles suspended in 100 μl of PBS into the dorsal flank SC tissue of the mice. Following injection, the Ig intronic enhancer element is activated, resulting in introduction of point mutations in the host DNA up to about 2 kb downstream of the enhancer. Where the point mutations are oncogenic, tumors form within about 3 to 4 months of injection. The mice with identifiable tumors are sacrificed and the tumors are excised.

The DNA from the tumor cells is isolated, and the mutated gene of interest is then identified by amplification from the isolated DNA (e.g., by PCR). The enhancer element is used as a “tag” to identify the mutated gene. The primers used in the PCR step are complementary to the enhancer element. This allows the selective amplification of the gene of interest by exponentially amplifying the fragment with the enhancer element complementary to the PCR primer. The mutated gene (flanking the enhancer element) is isolated from the other DNA after amplification, then is sequenced using techniques well known in the art. BLAST searching is performed to locate the mutated gene. The location of the genetic variation indicates the presence of an oncogene. The sequence of the mutated gene is compared to the wild-type sequence to determine the location of the point mutations.

### Example 2

Tumorigenic Point Mutations

Methods

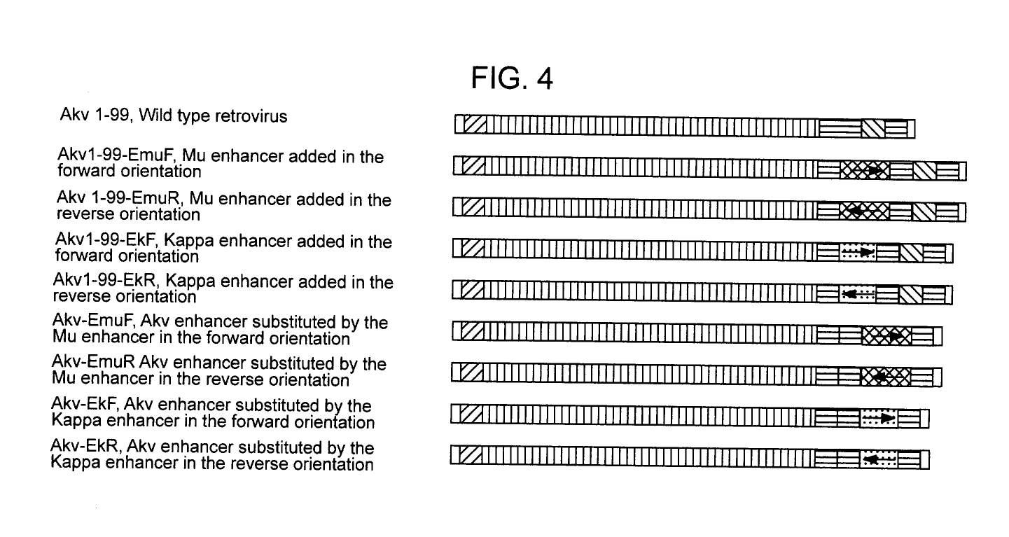

Eight transgenic viruses were engineered to deliver a cis-acting hypermutation element. All viruses were based on the replication competent Akv1-99 murine leukemia virus. The transgenic Akv1-99 viruses carried either the kappa (light chain) or mu (heavy chain) immunoglobulin intronic enhancers (SEQ ID NOS:2 and 1, respectively, as provided above), either in place of or in addition to the wild-type Akv enhancer. To account for differences arising from enhancer orientation, the enhancers were inserted in both a forward and reverse orientation (FIG. 4). The enhancer modifications to the Akv1-99 retrovirus were engineered into the U3 region of the 3′ LTR on a plasmid carrying the Akv1-99 genome. Transgenic virus designations were as follows:

| | |
|---|---|
| Akv1-99-EmuF | Mu enhancer added in the forward orientation |
| Akv1-99-EmuR | Mu enhancer added in the reverse orientation |
| Akv1-99-EkF | Kappa enhancer added in the forward orientation |
| Akv1-99-EkR | Kappa enhancer added in the reverse orientation |
| Akv1-EmuF | Akv enhancer substituted by the Mu enhancer |
| | in the forward orientation |
| Akv1-EmuR | Akv enhancer substituted by the Mu enhancer |
| | in the reverse orientation |
| Akv1-EkF | Akv enhancer substituted by the Kappa enhancer |
| | in the forward orientation |
| Akv1-EkR | Akv enhancer substituted by the Kappa enhancer |
| | in the reverse orientation |

The plasmid constructs containing the viral sequences were transfected by method of calcium phosphate precipitation into BOSC23, a Moloney retroviral packaging cell line. Viral particles were harvested from the media supernatant of the transfected culture and injected into newborn inbred NMRI mice. Wild-type Akv1-99 and sterile media (mock injection) were used as controls. Each virus or control injection was administered to between 10 and 20 mice. After sufficient viral incubation, mice were euthanized and tumors were excised. Genomic DNA was extracted from the tumors and DNA flanking the viral integration sites was amplified by anchored PCR. The PCR products were cloned into plasmids and sequenced. Genes and mutations in the virally tagged tumor DNA were identified by sequence comparison with the mouse genome of the Celera database.

Results and Discussion

In mice infected with virus carrying immunoglobulin enhancers, tumors were observed after approximately 5 months, while in mice infected with the wild-type virus, tumors were observed after approximately 7 months. The immunoglobulin enhancer containing viruses induced tumors primarily of the spleen and lymph nodes, while the wild-type virus induced tumors of the spleen, lymph node, and thymus. In the mice injected with viruses, >95% of the mice developed tumors. Of the ten mock injected mice, one mouse developed a tumor after 7 months.

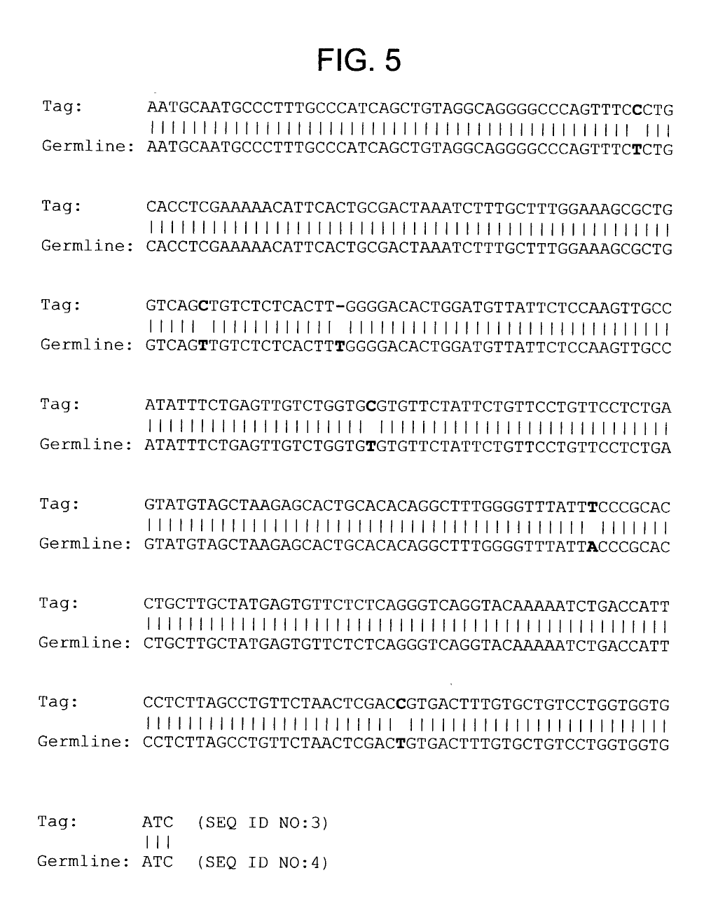

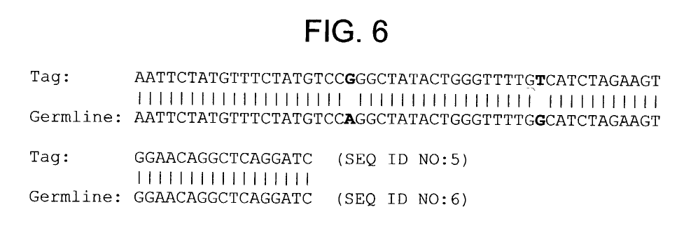

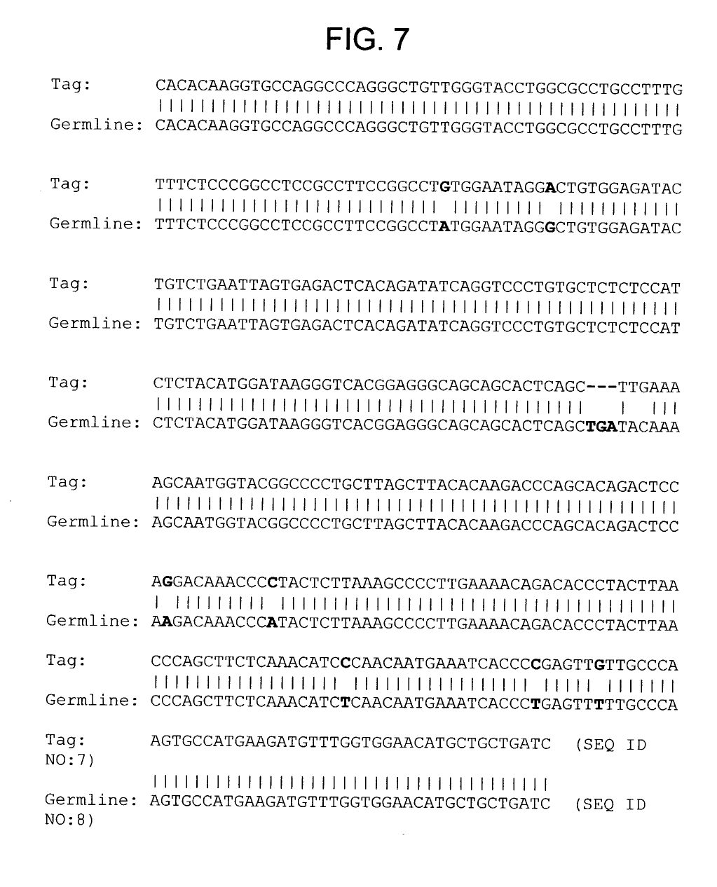

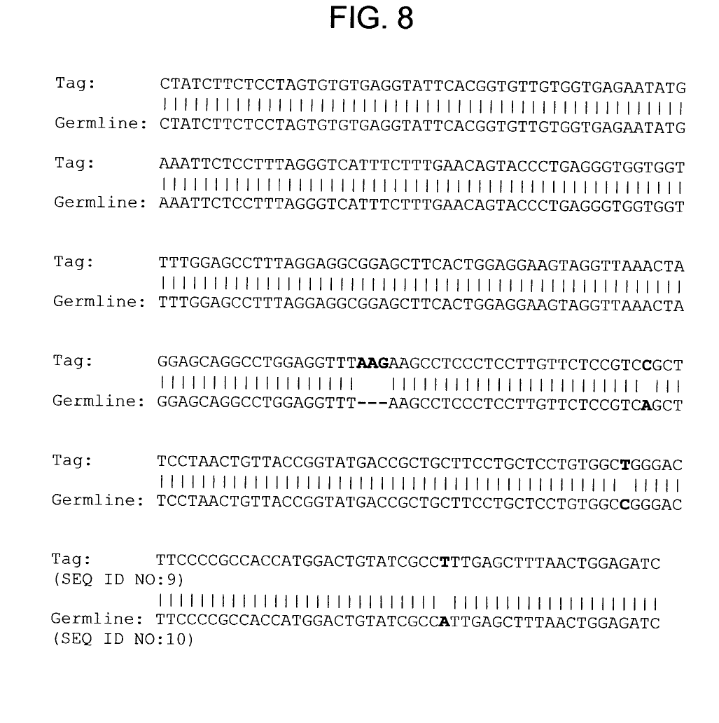

The integration sites of four tumors were analyzed. Of two splenic tumors from two mice infected with Akv1-99-EkF, one contained 11 integration sites and the other 8. Of two splenic tumors from two mice infected with Akv1-99-EmuF, one contained 5 integration sites and the other 3. Sequence analysis of DNA flanking the integration sites revealed that, in one tumor infected with Akv1-99-EkF, the sequence adjacent to one of the viruses contained of 2% point mutations (FIG. 5). In one tumor infected with Akv1-99-EmuF, 3 of 5 integration sites flanked sequences containing 2-3% mutations (FIGS. 6-8). In comparison, flanking sequences tagged by wild-type Akv1-99 typically have a mutation rate of <1% and often 0%. The increased mutation rate in genes adjacent to the transgenic viruses indicates that the virus is capable of inducing point mutations in genes flanking the viral integration site.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method for mutating a gene in a host cell genome, the method comprising:
introducing a hypermutation-inducing construct into a vertebrate host cell, the construct comprising a cis-acting hypermutation element;
wherein said introducing provides for integration of at least the cis-acting hypermutation element of the construct into a host cell genome and adjacent an endogenous host cell gene so that transcription of the endogenous host gene and the cis-acting hypermutation element facilitates introduction of a mutation into the endogenous host gene to generate a mutated gene.

2. The method of claim 1, wherein said hypermutation inducing construct comprises at least one immunoglobulin intronic enhancer.

3. The method of claim 2, wherein said immunoglobulin intronic enhancer is a heavy chain large intronic enhancer or a kappa intronic enhancer.

4. The method of claim 1, wherein the method further comprises identifying the mutated gene adjacent the cis-acting element.

5. The method of claim 4, wherein the mutated gene is identified by detecting all or a portion of integrated construct.

6. The method of claim 5, wherein the mutated gene is identified by amplification of at least a portion of a genomically integrated portion of the construct and a portion of the adjacent mutated gene to produce an amplification product comprising a sequence of the adjacent mutated gene.

7. The method of claim 1, wherein the mutated gene is associated with a cellular phenotype of interest.

8. The method of claim 7, wherein the phenotype of interest is an oncogenic phenotype.

9. The method of claim 1, wherein the host cell is a cultured cell or cell line.

10. The method of claim 1, wherein the host cell is in a non-human animal.

11. The method of claim 10, wherein the non-human animal is a murine non-human animal.

12. A method for identifying a proto-oncogene, which gene becomes oncogenic upon introduction of a mutation, the method comprising:
introducing a hypermutation inducing construct into a non-human animal host, or into any animal cell, including human, the construct comprising a cis-acting hypermutation element, wherein said cis-acting hypermutation element comprises an immunoglobulin intronic enhancer, said introducing providing for integration of at least the cis-acting hypermutation element into a host cell genome and adjacent an endogenous host gene so that transcription of the endogenous host gene and the cis-acting element facilitates production of a mutated host gene having a mutation;
detecting tumor formation in the host; and
identifying the mutated gene adjacent the cis-acting hypermutation element in nucleic acid of the tumor;
wherein detection of a tumor having a mutated endogenous gene adjacent the hypermutation element indicates that the endogenous gene is a proto-oncogene.

13. The method of claim 12, wherein said immunoglobulin intronic enhancer is a heavy chain large intronic enhancer or a kappa intronic enhancer.

14. The method of claim 12, wherein said hypermutation inducing construct is contained in a viral vector.

15. The method of claim 14, wherein said viral vector is a retroviral vector.

16. The method of claim 12, wherein the mutated gene is identified by detecting all or a portion of integrated construct adjacent the mutated gene.

17. The method of claim 12, wherein said mutated gene is identified by amplification of at least a portion of a genomically integrated portion of the construct and a portion of the adjacent mutated gene to produce an amplification product comprising a sequence of the adjacent mutated gene.

18. The method of claim 12, wherein the mutation is a point mutation.

19. A method for identification of a gene that becomes oncogenic after introduction of a mutation, the method comprising:
introducing a hypermutation inducing construct into a murine host, wherein the construct comprises a cis-acting hypermutation element, and wherein said cis-acting hypermutation element comprises a heavy chain large intronic enhancer or a kappa intronic enhancer, said introducing providing for integration of at least the cis-acting hypermutation element into a murine cell genome and adjacent an endogenous gene so that transcription of the endogenous gene and the cis-acting element facilitates production of a mutated gene having a mutation;
detecting tumor formation in the host; and
identifying the mutated gene adjacent the cis-acting hypermutation element in nucleic acid of the tumor;
wherein detection of tumors formed as a result of mutation of the endogenous gene indicates that the endogenous gene is a proto-oncogene.

20. The method of claim 19, wherein said hypermutation inducing construct is contained in a viral vector.

21. The method of claim 20, wherein said viral vector is a retroviral vector.

22. The method of claim 21, wherein said retroviral vector is derived from Mouse Mammary Tumor Virus or Murine Leukemia Virus.

23. The method of claim 19, wherein said identifying is by detecting an integrated portion of the integrated construct.

24. The method of claim 19, wherein said identifying is by amplification of at least a portion of a genomically integrated portion of the construct and a portion of the adjacent mutated gene to produce an amplification product comprising a sequence of the adjacent mutated gene.

25. A vertebrate cell having a genomically integrated cis-acting hypermutation element, which element is adjacent and operably linked to a gene endogenous to the vertebrate cell and with which the element is not normally found in nature.

26. The cell of claim 25, wherein the hypermutation element is adjacent a gene other than an immunoglobulin gene.

27. The cell of claim 25, wherein the hypermutation element is adjacent a gene other than a reporter gene.

28. The cell of claim 25, wherein the cell is a cultured cell or cell line.

29. The cell of claim 25, wherein the cell is a mutator positive cell.

30. An isolated vertebrate cell containing a mutated gene produced by the method of claim 1.

31. A vertebrate, non-human cell containing a mutated gene produced by the method of claim 1.

32. A non-human animal having a genomically integrated cis-acting hypermutation element, which element is adjacent and operably linked to a gene endogenous to the animal and with which the element is not normally found in nature.

33. The non-human animal of claim 32, wherein the hypermutation element is adjacent a gene other than an immunoglobulin gene or a reporter gene.

34. A vector comprising a cis-acting hypermutation element, wherein the vector is adapted for integration into a genome of a vertebrate cell, with the proviso that the cis-acting hypermutation element is not operably linked to a gene encoding a reporter polypeptide or a immunoglobulin polypeptide.

35. The vector of claim 34, wherein the vector is adapted for random integration in the vertebrate cell genome.

36. The vector of claim 34, wherein the vector is a viral vector.

37. The vector of claim 36, wherein the vector is a retroviral vector.

38. The vector of claim 34, wherein the cis-acting hypermutation element is an immunoglobulin intronic enhancer.

39. The vector of claim 38, wherein the immunoglobulin intronic enhancer is a heavy chain intronic enhancer or a kappa chain intronic enhancer.
